(19) United States
(12) Patent Application Publication
(10) Pub. No.: US 2025/0003941 A1
(43) Pub. Date: Jan. 2, 2025

(54) WASTE WATER ANALYSIS SYSTEM, METHOD FOR INCREASING THE SENSITIVITY OF THE WASTE WATER ANALYSIS SYSTEM, METHOD FOR INCREASING THE SENSITIVITY OF THE WASTE WATER

(71) Applicants: Endress+Hauser Conducta GmbH+Co. KG, Gerlingen (DE); Analytik Jena GmbH+Co. KG, Jena (DE)

(72) Inventors: Jennifer Führ, Jena (DE); Lars Bondzio, Gross-Umstadt (DE); Robert Möller, Jena (DE); Achim Gahr, Goldbach (DE); Jens Schoth, Essen (DE)

(21) Appl. No.: 18/756,364

(22) Filed: Jun. 27, 2024

(30) Foreign Application Priority Data
Jun. 30, 2023 (DE) 10 2023 117 307.3

(51) Int. Cl. G01N 33/18; G01N 21/33

(52) U.S. Cl. CPC G01N 33/18; G01N 21/33

(57) ABSTRACT

The present disclosure relates to a method for increasing the sensitivity of a wastewater analysis system for a primary analyte in a total sample of wastewater having a primary analyte and a secondary analyte. The method steps include steps of determining a primary analyte concentration of the primary analyte using a detection device that is suitable for determining a primary analyte concentration of the primary analyte. The primary analyte concentration correlates with a feces concentration and/or an ammonium concentration. A secondary analyte concentration of the secondary analyte is determined using a sensor. A sampler is suitable for taking a plurality of subsamples from the wastewater and storing the subsamples, and a computing unit providing sequential collection of the plurality of subsamples from the wastewater by the sampler at predetermined times over a predetermined period of time.

### CROSS-REFERENCE TO RELATED APPLICATION

The present application is related to and claims the priority benefit of German Patent Application No. 10 2023 117 307.3, filed on Jun. 30, 2023, the entire contents of which are incorporated herein by reference.

### TECHNICAL FIELD

The present disclosure relates to a method for collecting wastewater samples, to a method for increasing the sensitivity of a wastewater analysis system, and to a wastewater analysis system.

### BACKGROUND

In analytical measurement technology, especially in the fields of water management, of environmental analysis, in industry, e.g. in food technology, biotechnology, and pharmaceutics, as well as for the most varied laboratory applications, measured variables, such as the pH, the conductivity, or even the concentration of analytes, such as ions or dissolved gases in a gaseous or liquid measurement medium, are of great importance. These measured variables can be acquired and/or monitored for example by means of electrochemical sensors, such as optical, potentiometric, amperometric, voltammetric, or coulometric sensors, or also conductivity sensors. So-called analyzers are used to monitor a process medium regularly and fully automatically.

An “analyzer” in the sense of this present disclosure shall mean a measuring apparatus in process automation technology that measures, by means of a wet-chemical method, certain substance contents such as, for example, the ion concentration in a medium that is to be analyzed. For this purpose, a sample is taken from the medium to be analyzed. Usually, the taking of the sample is performed by the analyzer itself in a fully automated fashion by means of, for example, pumps, hoses, valves, etc. For determining the substance content of a certain species, reagents that were developed specifically for the respective substance content and that are available in the housing of the analyzer are mixed with the sample that is to be measured. A color reaction of the mixture caused thereby is subsequently measured by an appropriate measuring device, such as, for example, a photometer. Thus, the measured value is determined by the receiver, based upon light absorption and a stored calibration model.

A measurement system consisting of an analyzer and sampler is usually operated such that the sampler collects samples from the wastewater system at high frequency and charges mixed samples into a bottle tray over a defined period (e.g., 1 h or over 24 h), thus mixing and storing them in a stable manner by means of cooling. The wastewater sample is concentrated for the biomolecular wastewater analysis, and then impurities are eliminated or a further concentration by purification is carried out.

The extract obtained by concentration and purification is then checked for the presence of nucleic acids of the target organism preferably in the laboratory by means of PCR technology. Alternatively, the sample can be subjected to other analysis methods.

The problem here is, however, that an analysis in the laboratory is very time-consuming and cost-intensive/material-consuming (due to expensive reagents, consumables, disposable tools) and therefore cannot be carried out at the same frequency as sampling by the analyzer. For this reason, as mentioned above, the samples taken from the medium are mixed, whereby, for example, a sample period of 24 h is mapped by 24 mixed samples. In the case of low analyte concentrations in the medium, however, it can happen that the present analyte concentration is below the feasible limit of detection of the microbiological analysis (PCR) or of the analysis system

### SUMMARY

It is therefore an object of the present disclosure to propose a method which allows for the reliable, time-saving and cost-effective detection of the analyte even at low analyte concentrations in a simple and efficient manner.

This object is achieved according to the present disclosure by a method for increasing the sensitivity of a wastewater analysis system for a primary analyte in a total sample of wastewater having a primary analyte and a secondary analyte according to claim 1.

The method according to the present disclosure includes a step of providing a wastewater analysis system. The wastewater analysis system includes a detection device suitable for determining a primary analyte concentration of the primary analyte, wherein the primary analyte concentration correlates completely or partially with a feces concentration and/or an ammonium concentration. A sensor is suitable for determining a secondary analyte concentration of the secondary analyte. A sampler is suitable for taking a plurality of subsamples from the wastewater and storing the subsamples. The wastewater analysis system also includes a computing unit.

The method includes steps of sequentially collecting the plurality of subsamples from the wastewater by the sampler at predetermined times over a predetermined period of time, storing the subsamples, and determining of a secondary analyte concentration for the respective subsample by the sensor. The method also includes storing of the determined secondary analyte concentration of the respective subsample by the computing unit, evaluating of the determined secondary analyte concentrations by the computing unit, wherein secondary analyte concentrations that are within a predetermined region are determined, selecting the stored subsamples which have a secondary analyte concentration in the predetermined region to form the total sample, and determining of the primary analyte concentration of the total sample by the detection device.

The method according to the present disclosure allows for a reliable detection of the analyte to be possible even with low analyte concentrations present in the medium. Effort and costs are saved since only one sample has to be analyzed. The method further minimizes the sampling effort and logistics and thus saves costs. The method thus improves the limit of detection of the overall system of the workflow.

According to one embodiment of the present disclosure, the primary analyte is a SARS-CoV-2 virus, a Norovirus, an influenza virus, an antibiotic-resistant bacterium, a drug metabolite, a hormone or a biological particle and/or an organism or fragments thereof excreted via stool and/or urine.

According to a further embodiment of the present disclosure, the predetermined time period is one day.

According to one embodiment of the present disclosure, the evaluation step includes an integration of the secondary analyte concentration, wherein during the selection step takes place a selection of a greatest subintegral, and a selection of the subsample corresponding to the largest subintegral as a total sample takes place.

According to one embodiment of the present disclosure, the predetermined region comprises secondary analyte concentrations which deviate by less than 10%, preferably less than 5%, from a maximum secondary analyte concentration and are sequentially close to the maximum secondary analyte concentration or sequentially close to a selected secondary analyte concentration.

According to one embodiment of the present disclosure, the predetermined region is selected by an artificial intelligence and/or algorithms stored in the memory of the computing unit.

According to one embodiment of the present disclosure, the predetermined region comprises such secondary analyte concentrations that are greater than a predetermined threshold value.

The aforementioned object is also achieved by a wastewater analysis system according to claim 8.

The wastewater analysis system according to the present disclosure comprises a detection device suitable for determining a primary analyte concentration of a primary analyte, a sensor suitable for determining a secondary analyte concentration of a secondary analyte, a sampler suitable for taking a plurality of subsamples from the wastewater, storing the subsamples, and mixing specific subsamples to form a total sample, and a computing unit for storing and evaluating measurement data of the detection device and of the sensor.

According to one embodiment of the present disclosure, the computing unit has a digital communication unit which is suitable for being connected to a web-based database system.

The aforementioned object is also achieved by a method for sampling wastewater having a primary analyte and a secondary analyte according to claim 10.

The method according to the present disclosure includes sequentially collecting subsamples of the wastewater, determining a secondary analyte concentration for the respective subsample, evaluating the determined secondary analyte concentrations, wherein secondary analyte concentrations that are within a predetermined region are determined, selecting the subsamples which have a secondary analyte concentration in the predetermined region to form a total sample, and determining the primary analyte concentration of the total sample.

### BRIEF DESCRIPTION OF THE DRAWINGS

The present disclosure is explained in more detail on the basis of the following description of the figures. In the figures:

FIG. 1 shows a schematic image of a wastewater analysis system according to the present disclosure;

FIG. 2 shows a diagram of an ammonium content measured in the medium compared to a SARS-COV-2 concentration.

### DETAILED DESCRIPTION

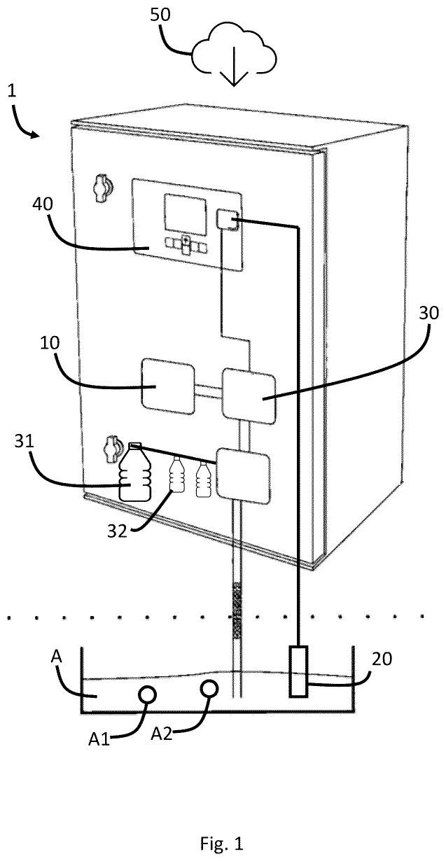

The wastewater analysis system 1 according to the present disclosure shown by way of example in FIG. 1 comprises a detection device 10, a sensor 20, a sampler 30, a computing unit 40, and a database 50.

The wastewater analysis system 1 is fluidly supplied with wastewater A. The wastewater A has a primary analyte A1 and a secondary analyte A2. For example, the wastewater analysis system 1 is connected via a hose to a wastewater tank or wastewater channel of a sewage treatment plant or manure pit. The wastewater A comprises human and/or animal feces and/or urine. FIG. 1 shows the wastewater analysis system 1 as a central unit. However, it is also possible for different components of the wastewater analysis system 1 to be arranged remotely.

The detection device 10 is suitable for determining a primary analyte concentration AK1 of the primary analyte A1. The primary analyte A1 is preferably SARS-COV-2. In an alternative embodiment, the primary analyte A1 is a different virus or organism. The primary analyte A1 is, for example, an influenza virus, Norovirus, antibiotic-resistant bacterium, drug, drug metabolite, or radioactively contaminated agricultural fertilizer. The primary analyte A1 is, for example, a nucleic acid of a pathogen (e.g., virus, bacterium) or of another biological particle or organism, the concentration of which in the wastewater is of interest. The biological particle or organism to be detected can be present in the wastewater as a whole or only in fragments or only as genetic material. The primary analyte A1 comprises, for example, hormones or drugs.

The detection device 10 preferably comprises a filtration unit for concentrating the primary analyte A1. The detection device 10 is suitable for conducting wastewater through the filtration unit by means of compressed air and/or mechanical pressure. The filtration unit is suitable for separating a liquid phase by static interactions or size exclusion. For example, the filtration unit comprises a cellulose mixed ester membrane. The filtration unit further comprises a purification unit, for example magnetic silicate beads. The purification unit is suitable for carrying out a concentration of the primary analyte, for example nucleic acids, and a removal of impurities. A PCR technology is preferably used for detecting the primary analyte A1. A different technology sensitive to the corresponding analyte can also be used instead of PCR technology. The detection device 10 is, for example, spatially separated from the sensor 20, the sampler 30, and the computing unit 40 (not shown). The detection device 10 is located, for example, in a laboratory. Of course, it is also possible for the detection device 10 to be directly coupled to one another together with the sensor 20, the sampler 30, and the computing unit 40.

The sensor 20 is suitable for determining a secondary analyte concentration AK2 of the secondary analyte A2. The secondary analyte A2 is, for example, ammonium or another fecal marker, for example the chemical oxygen demand (COD). The sensor 20 is, for example, an optical sensor, preferably a photometric sensor, for example a UV absorption sensor.

The sampler 30 is suitable for taking a plurality of subsamples 32 from the wastewater A. The sampler 30 is moreover suitable for storing the subsamples 32 taken from the wastewater A, for example in a cooled container, so that the subsamples 32 remain chemical/biologically/physically stable. The sampler 30 is also suitable for selecting subsamples 32 and mixing these specific subsamples 32 to form a total sample 31. The sampler 30 is also suitable for automatically discarding collected samples/subsamples according to predefined limit values or time intervals and thus to initialize fully automatically for a new sampling cycle. Alternatively, the mixing of the subsamples or the discarding of the samples can of course also be done manually.

The computing unit 40 preferably has a memory and is suitable for storing and evaluating measurement data from the detection device 10 and sensor 20. The computing unit 40 is preferably also suitable for controlling the detection device 10, the sensor 20, and the sampler 30. The computing unit 40 is, for example, a microcontroller or another computing unit. The computing unit 40 preferably has a digital communication unit which is suitable for being connected to the web-based database system 50 in order to transmit, store, and graphically provide the obtained measurement results and status information of the wastewater analysis system 1 continuously, i.e., in real time, to users. The computing unit 40 and/or the web-based database system 50 are suitable, for example, for generating alarm messages in real time and sending them to the users in order to inform the user about the exceedance of limit values or about the occurrence of the analyte A1.

The method according to the present disclosure for increasing the sensitivity of the wastewater analysis system 1 for a primary analyte A1 in the total sample 31 from wastewater A is described below.

The method comprises at least the following steps:

In a first step, the wastewater analysis system 1 described above is provided. This means that the wastewater analysis system 1 is supplied with wastewater A and is ready for operation.

In a next step, the plurality of subsamples 32 are sequentially taken from the wastewater A by the sampler 30. The subsamples 32 are collected at predetermined times T1, T2. They are collected, for example, every hour or every 10 minutes. Collection takes place over a predetermined period T, for example over one day. The subsamples 32 have, for example, a volume of 100 ml to 50 liters. Preferably, a volume-proportional sampling is carried out. Depending on the volume of the total flow of the sample source, for example the sewage treatment plant, the volume of the subsample is defined proportionally to this value. Individual sampling takes place in a few seconds, for example by sucking in the sample or by a compressed air system. The subsamples 32 are filled into containers, for example bottles arranged in a bottle tray, by the sampler 30. The containers are arranged, for example, in a type of carousel with 24 bottles, which are filled sequentially. Thus, a bottle tray with, for example, 24 bottles represents one day. Of course, it is also possible to provide more or fewer containers for receiving the subsamples 32. The subsample 32 is preferably numbered or filled into numbered containers.

Next, the subsamples 32 are stored. Preferably, the subsample 32 are stored in the sampler 30 such as to be cooled. This makes it possible for the subsamples 32 to remain stable.

A step of determining a secondary analyte concentration AK2 for the respective subsample 32 is then carried out by the sensor 20. Preferably, the determination step takes place at the same time as sampling. The concentration of ammonium (NH4-N) is determined by means of photometric analysis by the sensor 20, wherein a reagent is metered into the respective subsample 32, which results in a change in color depending on the NH4-N concentration. This change in color is then measured by the sensor, in particular a photometer. Including a sample with known NH4-N concentration can linearize the measuring system which thus allows for determining the NH4-N concentration of the sample. Said linearization takes place as follows, in detail: first, a raw value absorption is determined for an included reference. The included reference is a known NH4-N concentration. A raw value of an absorption of a reference having a zero concentration (e.g., cleaning solution) is then determined. By linear correlation between determined absorption and known NH4-N concentration, the application of a first-order function can be (linearly) applied.

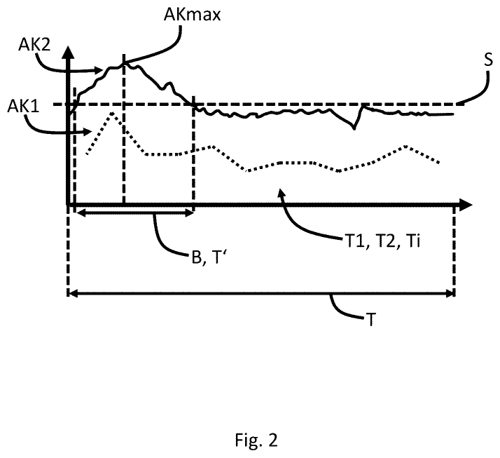

In a next step, the determined secondary analyte concentration AK2 of the respective subsample 32 is stored by the computing unit 40. The secondary analyte concentration AK2 determined for each subsample 32 is stored here in the memory of the computing unit 40 for later evaluation. FIG. 2 shows on the basis of the curve, which is represented by a full line, an exemplary development of the secondary analyte concentration AK2 over 24 hours. In the example shown in FIG. 2, the secondary analyte concentration AK2 was not measured discretely, but quasi-continuously, i.e., in very short time intervals between sampling times Ti. However, the curve appears as a continuous curve since the measurement points were connected to one another for illustration purposes.

A step of evaluating the determined secondary analyte concentrations is then carried out by the computing unit 40. This step preferably takes place at the end of each day, i.e., after 24 h-subsamples 32 have been taken from the wastewater A and filled into a container. The evaluation step preferably includes determining a maximum secondary analyte concentration AKmax from all secondary analyte concentrations AK2 of all subsamples 32 (see FIG. 2). In addition, for example all secondary analyte concentrations AK2 which are arranged in a predetermined region B around the maximum secondary analyte concentration AK2 are determined.

According to an alternative embodiment, the evaluation step includes integrating the ammonium concentration curve between the time intervals of wastewater sampling, followed by selecting the greatest subintegral and hence selecting the most relevant wastewater subsample. Such evaluation makes sense in particular in case of time-proportional sampling. In case of volume-proportional sampling, an additional multiplication of the subintegrals with respective flow volume flows is carried out. This results in a load value (i.e., an integration of the ammonium loads). Subsequently, the largest ammonium load subintegral and thus the corresponding most relevant wastewater sample is selected.

The predetermined region B preferably comprises secondary analyte concentrations AK2, which deviate by less than 10%, preferably less than 5%, from the maximum secondary analyte concentration AKmax and are sequentially close to the maximum secondary analyte concentration AKmax or sequentially close to a selected secondary analyte concentration AK2. “Sequentially close” is to be understood here to mean that a measured value, i.e., a determined concentration, was determined directly prior to or directly after another measured value.

According to an alternative embodiment, the predetermined region B is selected by an artificial intelligence stored in the memory of the computing unit 40. This means that the artificial intelligence is fed with the input variable: secondary analyte concentration AK2, time Ti of sampling, flow (in case of volume-proportional sampling), and further data, for example weather data and/or weather forecast data. The artificial intelligence then outputs the optimal region B, which denotes specific secondary analyte concentration AK2 or the associated subsamples 32. The artificial intelligence thus outputs, for example, the numbers of the subsamples 32 which fall into region B.

According to an alternative embodiment, the predetermined region B comprises such secondary analyte concentrations AK2 that are greater than a predetermined threshold value S. The threshold value S is, for example, the mean value between the highest measured secondary analyte concentration AK2 and the lowest measured secondary analyte concentration AK2.

In a next step, the subsample 32 associated with the maximum secondary analyte concentration AK2, and the subsamples 32 which have a secondary analyte concentration AK2 in the predetermined region B are selected. Since the subsamples 32 are preferably numbered, the computing unit 40 preferably determines the number of the subsamples 32.

In the event that several subsamples 32 were selected, i.e., lie in the predetermined region B, a step of mixing the selected relevant subsamples 32 by the sampler 30 to form a total sample 31 takes place. The total sample 31 is preferably filled into a container large enough to receive the subsamples 32, or is transferred from the containers of the subsamples 32 into the container of the total sample 31. The mixing step takes place automatically or manually, for example. The mixing step is particularly advantageous in the event of very small volumes, for example 100 ml, of the subsamples 32.

Furthermore, a step of determining the primary analyte concentration AK1 of the total sample 31 is carried out by the detection device 10. This step takes place, for example, in the wastewater analysis system 1. According to an alternative embodiment, however, this step can also take place geographically remotely from the other components present in the wastewater analysis system 1, for example in a laboratory. The detection device 10 preferably carries out a PCR analysis with the total sample 31, wherein nucleic acids are detected and quantified. As mentioned above, it is also possible to use another analysis system for determining the primary analyte concentration AK1 if the primary analyte A1 is, for example, a drug metabolite.

FIG. 2 shows on the basis of the dotted curve an example of a profile of the primary analyte concentration AK1 that was determined from a plurality of sequential subsamples 32. However, no selection of the subsamples 32 on the basis of their secondary analyte concentration AK2 was carried out here.

As can be seen in FIG. 2, it was demonstrated that there is a correlation between the concentration of ammonium and the concentration of SARS-COV-2 viruses. Since SARS-COV-2 viruses are present in particular in feces, and since the presence of feces in wastewater A increase the concentration of ammonium, ammonium can be used as an indicator of the optimal sampling period or the optimal selection of wastewater samples.

Thanks to the present disclosure, the limit of detection of the overall system (consisting of sampling and sample processing and analysis) is reduced by the ammonium-dependent selection of the samples. The wastewater analysis system 1 is thus more sensitive, since samples having a high virus load are no longer mixed (as before) with samples having a low sample load before analysis by the detection device 10. Thanks to the selection of the samples in region B, a dilution or attenuation in concentration of the analyte to be analyzed is thus avoided if assuming that the analyte is not present at the same concentration at all times of day.

Furthermore, the present disclosure makes it possible that, as a result of sample processing and analysis of subsamples in the specific relevant region B, no sampling can theoretically take place over the entire day, but only in a reduced time period T′ if the daily concentration profile is almost identical, resulting in further effort and cost savings during sampling.

The logistical, manual, material, temporal, and cost effort associated with sample transportation, preparation, and analysis is reduced by the wastewater analysis system 1 according to the present disclosure.

Thanks to the present disclosure, it is possible to improve the limit of detection of the wastewater analysis system 1 and to avoid false epidemiological conclusions (such as a false negative ones).

1. A method for increasing the sensitivity of a wastewater analysis system for a primary analyte in a total sample of wastewater having a primary analyte and a secondary analyte, wherein the method comprises at least the following steps:
providing the wastewater analysis system, comprising:
a detection device suitable for determining a primary analyte concentration of the primary analyte, wherein the primary analyte concentration correlates completely or partially with a feces concentration or an ammonium concentration;
a sensor suitable for determining a secondary analyte concentration of the secondary analyte;
a sampler suitable for taking a plurality of subsamples from the wastewater and storing the subsamples;
a computing unit;
sequential collection of the plurality of subsamples from the wastewater by the sampler at predetermined times over a predetermined period of time;
storing the subsamples;
determining of a secondary analyte concentration for the respective subsample by the sensor;
storing of the determined secondary analyte concentration of the respective subsample by the computing unit;
evaluating of the determined secondary analyte concentrations by the computing unit, wherein secondary analyte concentrations that are within a predetermined region are determined;
selecting the stored subsamples which have a secondary analyte concentration in the predetermined region to form the total sample;
determining of the primary analyte concentration of the total sample by the detection device.

2. The method according to claim 1, wherein the primary analyte is a SARS-COV-2 virus, a Norovirus, an influenza virus, an antibiotic-resistant bacterium, a drug metabolite, a hormone or a biological particle and/or an organism or fragments thereof excreted via stool and/or urine.

3. The method according to claim 1, wherein the predetermined time period is one day.

4. The method according to claim 1, wherein the evaluation step includes an integration of the secondary analyte concentration, wherein the selection step takes place a selection of a greatest subintegral, and a selection of the subsample corresponding to the largest subintegral as a total sample takes place.

5. The method according to claim 1, wherein the predetermined region comprises secondary analyte concentrations which deviate by less than 10% from a maximum secondary analyte concentration and are sequentially close to the maximum secondary analyte concentration or sequentially close to a selected secondary analyte concentration.

6. The method according to claim 1, wherein the predetermined region is selected by an artificial intelligence or algorithms stored in the memory of the computing unit.

7. The method according to claim 1, wherein the predetermined region comprises such secondary analyte concentrations that are greater than a predetermined threshold value.

8. A wastewater analysis system, comprising:
a detection device suitable for determining a primary analyte concentration of a primary analyte;
a sensor suitable for determining a secondary analyte concentration of a secondary analyte;
a sampler suitable for taking a plurality of subsamples from the wastewater, storing the subsamples, and mixing specific subsamples to form a total sample; and
a computing unit for storing and evaluating measurement data of the detection device and of the sensor.

9. The wastewater analysis system according to claim 8, wherein the computing unit has a digital communication unit which is suitable for being connected to a web-based database system.

10. A method for sampling wastewater having a primary analyte and a secondary analyte, wherein the method comprises at least the following steps:
sequentially collecting subsamples of the wastewater;
determining a secondary analyte concentration for the respective subsample;
evaluating the determined secondary analyte concentrations, wherein secondary analyte concentrations that are within a predetermined region are determined;
selecting the subsamples which have a secondary analyte concentration in the predetermined region to form a total sample;
determining the primary analyte concentration of the total sample.
